(19) United States
(12) Patent Application Publication
(10) Pub. No.: US 2024/0018510 A1
(43) Pub. Date: Jan. 18, 2024

(54) METHODS FOR SEQUENCING POLYNUCLEOTIDE FRAGMENTS FROM BOTH ENDS

(71) Applicant: Agilent Technologies, Inc., Santa Clara, CA (US)

(72) Inventors: David Taussig, Sunnyvale, CA (US); Israel Steinfeld, Sunnyvale, CA (US); Nicholas M Sampas, San Jose, CA (US); Brian Jon Peter, Los Altos, CA (US)

(21) Appl. No.: 18/256,877

(22) PCT Filed: Dec. 10, 2020
(86) PCT No.: PCT/US2020/064297

§ 371 Date: Jun. 9, 2023

(51) Int. Cl. C12N 15/10; C12Q 1/6869

(52) U.S. Cl. CPC C12N 15/1065; C12Q 1/6869

(57) ABSTRACT

The present invention relates to preparation, sequencing and analysis of a sequencing library of adaptor-tagged fragments, wherein the fragments have different orientations relative to a sequencing adaptor.

### CROSS-REFERENCE TO RELATED APPLICATIONS

None.

### FIELD OF THE INVENTION

The present invention relates to preparation, sequencing and analysis of a sequencing library of polynucleotide fragments.

### BACKGROUND

Next-Generation Sequencing (NGS) methods and systems involve the parallel sequencing of a library of polynucleotide fragments by a sequencing system. Preparation of a sequencing library generally includes amplification of the polynucleotide fragments, attachment of adaptors, and/or other preparatory steps. An adaptor can be attached to one or both ends of the fragments in order to add sites for primer binding and other functional sequences to the fragments. Various kinds of adaptors are used in sequencing preparation kits to add these sites or sequences to the fragments from the sample. Adaptors can be attached in various ways, such as by ligation, primer extension, tagmentation, and other techniques.

In order to obtain a suitable signal from sequencing a single DNA fragment, many sequencing systems use clonal amplification to generate many identical copies of individual DNA molecules on a solid support. These copies are segregated in individual clusters, or on beads which are loaded with an individual DNA molecule. Sequencing reactions proceed on the identical copies of the fragment in parallel, thereby producing detectable signals from the clusters or beads, with signals simultaneously detected from an enormous number of distinct clusters or beads.

A sequencing library can be generated in a variety of ways, with different objectives regarding the fragments to be used as inputs. In amplicon sequencing, PCR is used to generate a library of amplicons covering regions of interest in the nucleic acid sample, targeted by specific primers. Other methods of library preparation involve random fragmentation of the nucleic acid sample by enzymatic or physical shearing methods, followed by amplification using common adapter sequences. In these random fragmentation methods, the genome can be sampled with less bias, but the beginning and end (start and stop) of each genomic fragment is not known until sequencing and alignment.

The most common applications for NGS in the sequencing of human genomic DNA involve alignment of the sequencing reads to a reference sequence (such as a reference genome) in order to identify aberrations in the sequenced genomic DNA. Aberrations of clinical significance include copy number variations, SNVs, and chromosomal rearrangements. Chromosomal rearrangements are typically identified by observing an increased rate of alignments sharing a common end, or by observing a single alignment linking separated regions of the genome. In either case, longer alignments increase the chance of detecting a chromosomal rearrangement. Longer alignments are particularly beneficial under conditions with low read depth, allele frequency, or library complexity. Since the genomic fragments being generated from a sample are often longer than the length of the sequencing read, various methods have been employed to increase the alignment length by utilizing the entire sequence of the fragment, rather than being limited by sequencing read length.

There are several methods currently being used to generate alignments of longer length than the sequencing reads themselves. The most popular is paired-end sequencing technology, such as that provided by sequencing systems from Illumina. This enables the analyst to link two reads originating from opposite ends of the same genomic fragment on the basis of their physical colocation on the sequencer flow cell, and thereby combine the reads into a single alignment. Paired-end reads are advantageous for several reasons. They generally allow one to obtain more sequence information from a single genomic fragment than allowed by a single-end read, since genomic fragments are generally longer than typical read lengths. Paired-end reads also allow an analyst to align a sequenced fragment to a greater length of a reference genome than the length of the sequencing read(s). This can be beneficial when measuring clinically relevant genomic aberrations such as translocations, deletions, and gene fusions. On Illumina's platforms, paired-end reading requires two sequential sequencing runs, where each sequencing run produces a read from a different end of the fragment. Another method is 10× Genomics' synthetic long read technology, which works by partitioning long genomic fragments into droplets prior to fragmenting and barcoding smaller fragments which are then sequenced. Reads can then be linked in silico through use of a common barcode assigned to all fragments within each partition. Other methods of generating alignment information for long fragments involve circularization of long genomic fragments by ligation, sequencing near the ligation junction, and generating long alignments by linking sequences from relatively distant (up to 50 Kb) regions of the genome.

Smith US 2009181370 discusses methods for pairwise sequencing of a double-stranded polynucleotide template, which methods are said to permit the sequential determination of nucleotide sequences in two distinct and separate regions on complementary strands of the double-stranded polynucleotide template. The two regions for sequence determination may or may not be complementary to each other. Rigatti et al. US 2009088327 also discusses methods for pairwise sequencing of a double-stranded polynucleotide template. Using the methods, it is said to be possible to obtain two linked or paired reads of sequence information from each double-stranded template on a clustered array, rather than just a single sequencing read from one strand of the template.

There remains a need for improved methods of sequencing polynucleotide fragments.

### SUMMARY

The present methods provide sequencing libraries comprising adaptor-tagged insert fragments in which an insert fragments present in two orientations with respect to a sequencing adaptor. The generation of dually-orientated insert fragments occurs in preparation of a sequencing library rather than on a flow cell or during a sequencing run. Further, the present methods provide the capability to pair multiple reads derived from the same input fragment but sequenced from opposite directions at different physical locations on the sequencing system.

The present methods are platform independent, and thus allows users to obtain ‘paired-end’ read information irrespective of their chosen NGS instrument. A second advantage of the present methods is decreased sequencing time relative to approaches utilizing sequential sequencing reads for paired-end sequencing.

The present methods can generate the ‘paired’ information with a single sequencing run of genomic sequence. In some embodiments, reads from separate sequencing runs can be paired, enabling an analyst to decide whether more sequencing or more pairing of a sequencing library is needed. In some embodiments where multiple MBCs are used, the present methods allow for sequencing from both strands which is helpful for redundancy/error reduction. Another benefit of such embodiments is that sequencing of both strands of each genomic fragment occurs, an advantage currently restricted to libraries generated with branched adaptors (e.g. Illumina's Y adaptor and NEB's hairpin adaptor). Sequencing both strands of a fragment is highly beneficial in calling extremely rare mutations, such as SNVs in ctDNA.

### BRIEF DESCRIPTION OF THE DRAWINGS

FIG. 1 illustrates an embodiment of the present methods in which amplicons or copies of tagged fragments are generated in which the insert sequence is inverted with respect to the sequencing adaptors.

FIGS. 2A and 2B illustrate embodiments of methods for generating a MBC pairing oligo.

FIGS. 3A and 3B illustrate other embodiments of methods for generating a MBC-pairing oligo.

FIG. 4 illustrates an embodiment of a method for generating a circularizing adaptor.

FIGS. 5A and 5B illustrates an embodiment of methods for generating a library with two orientations of adaptors relative to the sequence of the input fragment.

FIGS. 6A and 6B illustrate an embodiment of a method of sequencing a library of adaptor-tagged fragments following cluster generation on a solid surface of a sequencing system.

It is to be understood that the figures are for purposes of describing particular embodiments only, and are not intended to be limiting. The features in the figures are not intended to be drawn to scale. The present invention can be readily understood from the following detailed description when read with the accompanying figures.

### Definitions

“Orientation” of a polynucleotide sequence generally refers to whether the sequence is from 5′ to 3′, or from 3′ to 5′. When referring to a double-stranded polynucleotide, the term “orientation” can refer to the orientation of a top strand or a bottom strand, or it can refer to the sequence relative to one or more other points. For example, if two polynucleotide molecules have the sequence 5′-AATGCC-3′, but one is attached to an adaptor at its 5′ end and the other is attached to an adaptor at its 3′ end, the two polynucleotide molecules have different orientations relative to the adaptor. Alternatively, if a 5′ end of the complementary molecule (e.g., 5′-GGCATT-3′) is attached to an adaptor, these molecules also have a different orientation relative to the adaptor.

The term “inverted”, as used herein with respect to a nucleic acid sequence, means the sequence is reversed in position, order or relationship. For example, a sequence comprising 5′-AATGCC-3′ which is attached to a support at its 5′ end is inverted if the sequence is attached to a support at its 3′ end instead. Alternatively, a sequence is inverted if a 5′ end of its complement (e.g., 5′-GGCATT-3′) is attached to a support instead.

The terms ‘insert’ or ‘input fragment’ refer to the nucleic acid molecule of biological or synthetic origin whose sequence and/or alignment is the object of the sequencing reaction. The insert sequence does not include barcode, index, or adaptor sequences which may be added to the input fragment and/or its amplicons during library preparation or sequencing. Amplification does not change the insert sequence unless errors are introduced during the amplification step.

The term “sequencing read” or “read” refers to an experimentally determined sequence of a polynucleotide fragment from a sequencing run. A read is generally of sufficient length (e.g., at least about 20 nt) that can be used to identify a larger sequence or region, e.g. that can be aligned and specifically assigned to a chromosome location, genomic region, or gene.

A “sequencing run” refers to a series of physical or chemical steps that generate signals indicating the order of bases in a polynucleotide. The series of steps can be carried out until the generated signals no longer distinguish bases of the polynucleotide with a reasonable level of certainty. Alternatively, the series of steps can be stopped earlier, for example, once a desired amount of sequence information has been obtained. A sequencing run can be carried out on a single polynucleotide fragment or simultaneously on a population of fragments having the same sequence, or simultaneously on a population of fragments having different sequences. For example, a sequencing run can be initiated for one or more adaptor-tagged fragments that are present on a solid support of a sequencing system, and terminated upon removal of the one or more adaptor-tagged fragments from the solid support or otherwise ceasing detection of the adaptor-tagged fragments that were present on the solid support when the sequencing run was initiated.

The terms “aligned” or “alignment” refer to one or more sequences that are identified as a match in terms of the order of their nucleic acid molecules to a known reference sequence, such as a reference genome.

The term “reference sequence” means a previously identified nucleic acid sequence, which may be available in a database as an example of a species or subject for comparison.

The term “oligonucleotide” or “oligo” as used herein denotes a multimer of nucleotides of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers, or both ribonucleotide monomers and deoxyribonucleotide monomers.

The term “primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. Primers are generally of a length compatible with their use in synthesis of primer extension products, and are usually are in the range of between 8 to 100 nucleotides.

The term “amplifying” as used herein refers to the process of synthesizing nucleic acid molecules that are complementary to one or both strands of a template nucleic acid. Amplifying a nucleic acid molecule may include denaturing the template nucleic acid, annealing primers to the template nucleic acid at a temperature that is below the melting temperatures of the primers, and enzymatically elongating from the primers to generate an amplification product. The denaturing, annealing and elongating steps each can be performed one or more times. Amplification typically requires the presence of deoxyribonucleoside triphosphates, a DNA polymerase enzyme and an appropriate buffer and/or co-factors for optimal activity of the polymerase enzyme. The terms “amplicon” or “amplification product” refers to the nucleic acid sequences, which are produced from an amplifying process.

The terms “sequence tag” and “adaptor” generally refer to nucleic acid molecules that are attached to another nucleic acid molecule to add a desired structure or function. For example, a sequence tag can be attached to an input fragment to add a barcode or a primer binding site. As another example, an adaptor can be attached to an input fragment or an amplicon thereof to add a binding site for a NGS platform. In some embodiments, an adaptor refers to molecules that are at least partially double-stranded. An adaptor or a sequence tag may be any desired length, including but not limited to 40 to 150 bases in length, e.g., 50 to 120 bases, although adaptors and sequence tags outside of this range are envisioned.

The term “barcode” refers to a sequence of nucleotides used to identify the origin of a sequence. Barcodes may comprise sample indices or sample barcodes, where the same sequence is shared for all nucleic acids from a particular source, organism, or sample. Sample barcodes enable the mixing of nucleic acids from different samples in one sequencing run, as the different sample barcode sequences enable the correct assignment of sequencing reads to each sample. One, two, or more sample barcodes may be used. Barcode sequences also comprise molecular barcodes (MBCs) or unique molecular identifier sequences, which function to identify copies of individual templates. MBCs may comprise random nucleotides, known nucleotides, or a mixture of random and known nucleotides. MBCs enable more accurate sequencing by allowing error correction of sequences and more accurate estimation of the original number of templates. In some embodiments, a large number of MBCs is used (e.g., 100,000, 1 million, 1 billion, or more possible sequences) such that each template has a unique molecular barcode. In other embodiments, a smaller number of molecular barcodes is used, and the beginning or ending positions (or both) of the sequence read are used together with the molecular barcode to identify copies arising from a unique nucleic acid template. Molecular barcodes may be combined with sample barcodes, on the same or different portions of the target nucleic acid. Molecular barcodes may be added to one end of a nucleic acid template (e.g., the 5′ end of the + strand, and the 3′ end of the − strand in a duplex), or to both ends of a template (e.g., to both the 5; and the 3′ ends of both the + and the − strands of the duplex).

### DESCRIPTION OF EXEMPLARY EMBODIMENTS

Before the various embodiments are described, it is to be understood that the teachings of this disclosure are not limited to the particular embodiments described, and as such can, of course, vary. The section headings used herein are for organizational purposes only and are not to be construed as limiting the subject matter described in any way.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

All patents and publications, including all sequences disclosed within such patents and publications, referred to herein are expressly incorporated by reference.

Preparing a Sequencing Library with Inverted Insert Fragments

The present disclosure describes novel methods for preparing a sequencing library in such a way as to obtain sequence information equivalent to paired-end reads on a next generation sequencing (NGS) platform. The present methods improve the utility of the single-end sequencing data by generating alignments of lengths equal to the original inserts rather than being limited by sequencing read length. Additional advantages include error reduction for sequences read from both directions, and decreased sequencing time relative to read pairing methods requiring multiple sequential insert reads (e.g. on Illumina sequencers).

In some embodiments of methods described in this disclosure, adaptor-tagged fragments are prepared by amplifying tagged fragments using two different pairs of primers to add adaptor sequences. The sequence of the insert fragment is inverted in different amplicons (copies) produced by the amplification of the tagged fragment, thereby forming some adaptor-tagged fragments having inverted insert fragments or different orientations of the insert sequence relative to one or more adaptors, and some adaptor-tagged fragments having non-inverted insert sequences. The adaptor-tagged fragments are introduced to a sequencing system, and sequencing primers are introduced such that both orientations can be sequenced simultaneously. MBCs are simultaneously sequenced, and the sequencing data are analyzed to pair the sequence reads from each orientation of the insert fragment.

An important advantage of the present methods is that a MBC in one orientation can be paired with the reverse complement of that MBC in an inverted orientation. For example, the MBC sequence 5′ CCAACGGTTA may uniquely identify sequences arising from one template, while the MBC sequence 5′ TAACCGTTGG may indicate sequences from a completely different template, or sequences from the inverted orientation of the first template. Longer MBCs may be used to reduce the chance of the same MBC being applied to more than one template, therefore increasing the confidence of pairing MBCs with their reverse complements. In some embodiments, MBCs may be designed such that information about the orientation is embedded in the barcode sequence, and/or known nucleotides can be used adjacent to or within the MBC to indicate orientation. By designing appropriate adaptor, barcode, and primer sequences, both orientations can be efficiently sequenced in the same sequencing run.

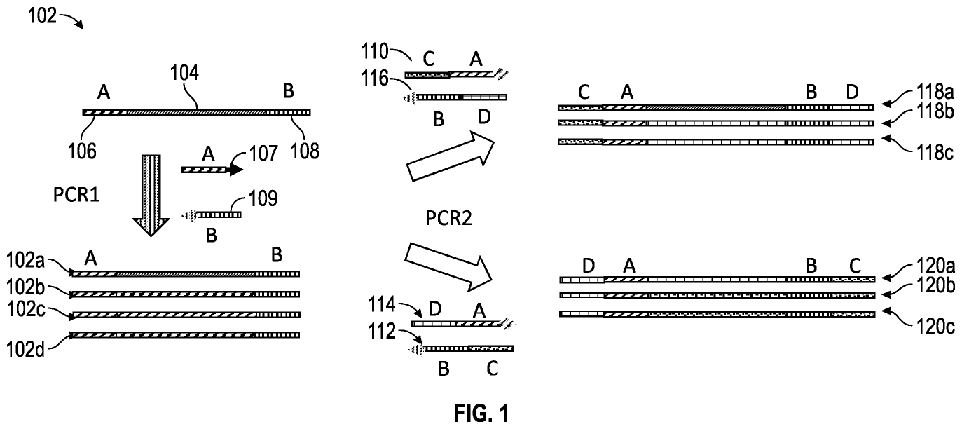

In some embodiments of the present methods, amplicons or copies of tagged fragments (such as tagged fragment 102) are generated in which the insert sequence is inverted with respect to the sequencing adaptors (FIG. 1). In some embodiments, this can be done with a two-stage amplification approach. Tagged fragment 102 is generated by attaching sequence tags 106 and 108 to each end of an insert fragment 104, such as by ligation. Sequence tag 106 comprises a first sequence (sequence A) and sequence tag 108 comprises a second sequence (sequence B), and at least one of sequence tags 106, 108 also contains a molecular barcode (not shown). The tagged fragments are then amplified in a first amplification stage with primers annealing to the sequence tags, more particularly to sequences A and B or portions thereof. In the first amplification stage, the tagged fragment 102 is amplified with a pair of primers 107, 109 which binds to sequences A and B, thereby generating many identical copies or amplicons 102a, 102b, 102c, 102d, which are also referred to herein as tagged fragments 102. For the second amplification stage, two parallel amplifications are carried with primer pairs 110 and 116 and separately with 112 and 114 to add sequence adaptors C and D to each end of the insert fragment, but with inverted orientations with respect to the insert sequence. Thus, multiple copies of fragment 118a, 118b, 118c are generated as well as inversely-oriented fragments 120a, 120b, and 120c, permitting sequencing of insert 104 from both directions. Alternatively, the parallel reactions of the second-stage amplification may be combined into a single reaction with all four primers. In other embodiments, amplicons with larger adapters can be generated in one orientation, and the orientation of the insert may be inverted in a subsequent PCR amplification. For example, the larger adapters which are initially ligated to the insert may comprise sequences C and D, in one orientation relative to the A and B sequences. For example, one adapter may comprise sequence C attached to sequence A and the second adapter may comprise sequence D attached to sequence B, such that after ligation and amplification with primers 110 and 116, fragments 118a, 118b, and 118c are generated. This would create a “forward orientation” library A which could already be sequenced. Subsequently or in parallel, this forward orientation library A could be diluted and re-amplified with primers 112 and 114, which would invert the insert to the inverted orientation B, and create fragments 120a, 120b, and 120c. An advantage of this embodiment is that an analyst would not need to decide whether to sequence the inverted orientation B until after the forward orientation library A is sequenced. Another advantage of this embodiment is that it may be possible to use fewer total cycles of amplification.

Methods for Pairing Insert Sequences with Two MBCs and Pairing Oligos

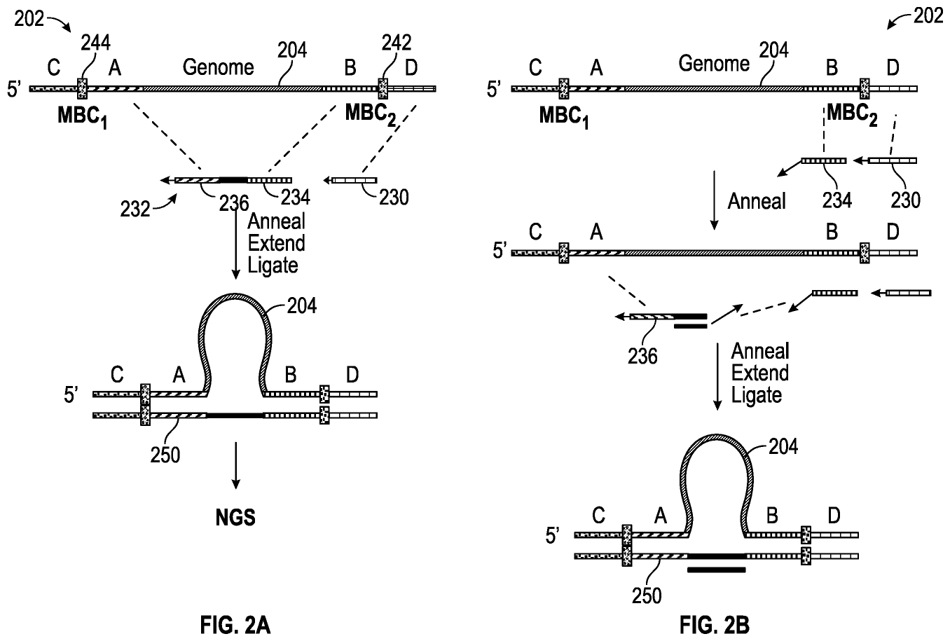

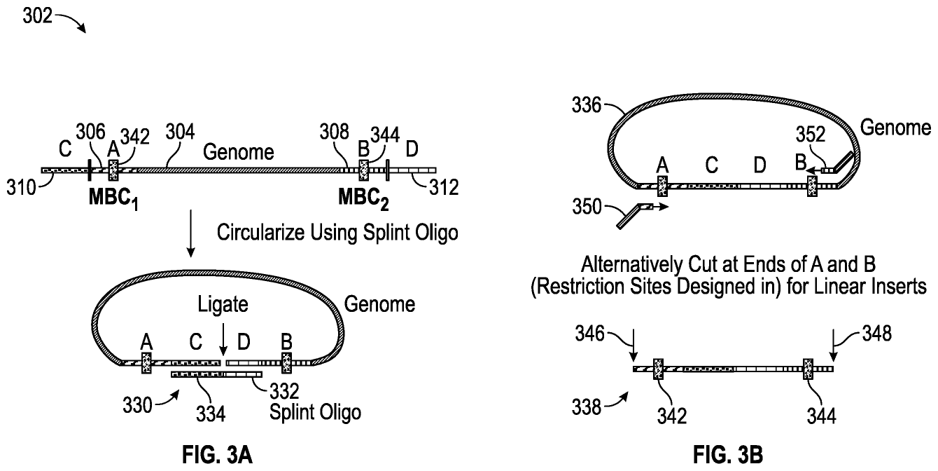

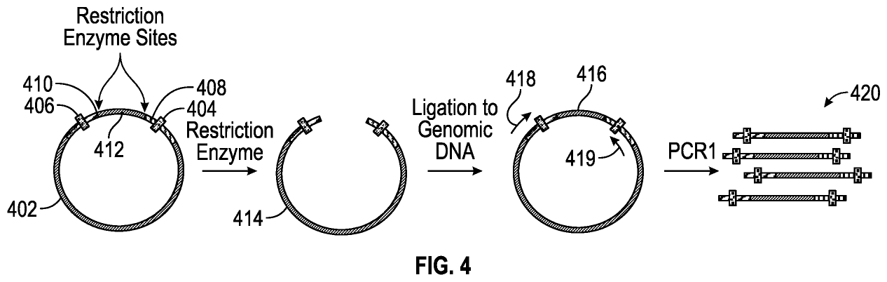

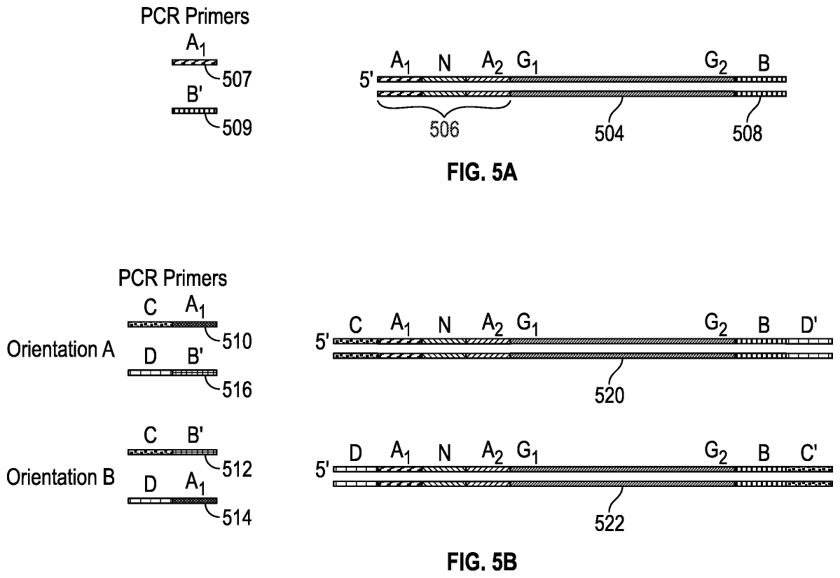

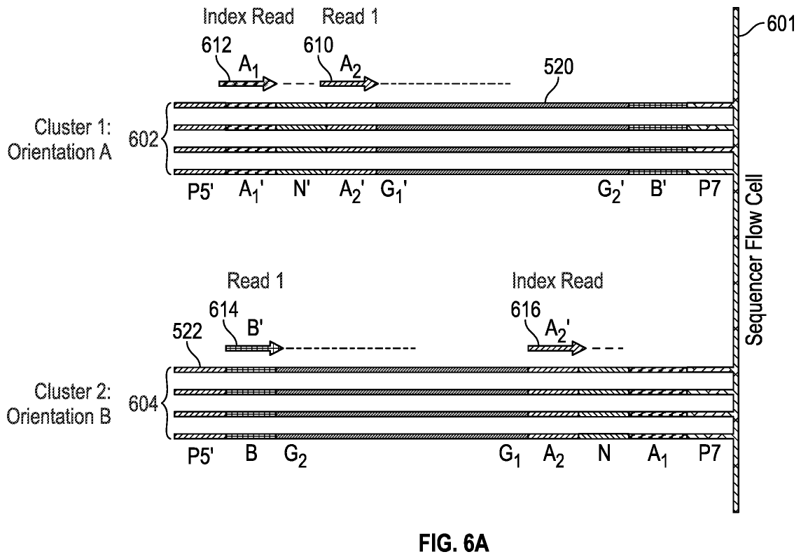

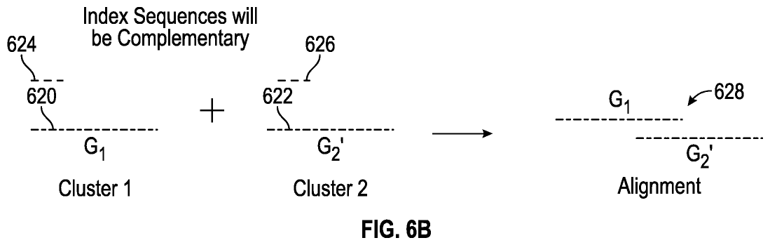

The adaptor-tagged fragments can be sequenced to generate sequence information from each end of the input fragment 104. In order to appropriately pair sequence reads belonging to opposite ends of the same input fragments, additional steps may be performed. In some embodiments (described in connection with FIGS. 2A to 3B), a sequence tag comprising a molecular barcode (MBC) is added to each end of an input fragment followed by generation of a MBC pairing oligo which can be sequenced to pair inversely-oriented insert reads on the basis of their MBC sequences. In other embodiments (described in connection with FIG. 4), the insert sequence is attached to a predetermined-pair of MBC sequences. In still other embodiments, (described in connection with FIGS. 5A to 6B), a sequence tag comprising a MBC is added on one end of an input fragment, and sequencing of the input fragment and the MBC can be used to pair sequence reads from inversely-oriented amplicons generated from the same input fragment.

FIGS. 2A and 2B illustrates how a MBC pairing oligo can be prepared from one of the copies of the adaptor tagged fragments 202. The adaptor-tagged fragments 202 contain molecular barcodes (MBCs) on both ends of each fragment 204. The adaptor-tagged fragments 202 are combined with an oligonucleotide 230 complementary to D and with an oligonucleotide 232 having a formula B′-X-A′. In oligo 232, the 3′ end 236 is complementary to A (interior to the MBC 244 of the 5′ adaptor), and the 5′ end 234 is complementary to B (interior to the MBC 242 of the 3′ adaptor). Following annealing of oligos 230 and 232 to fragment 202, oligos 230 and 232 are extended from their 3′ ends with a DNA polymerase. Oligo 230 is extended until it meets the 5′ end of oligo 232, then the extended oligos are ligated together with DNA ligase, generating a shorter sequenceable molecule 250 containing MBC information for MBCs 242 and 244 from both ends of a source input fragment 204. Sequencing of the pairing oligo 250 along with inversely-oriented amplicons of fragment 204 will permit pairing on the basis of their MBC sequences.

Another method for generating a MBC-pairing oligo is to circularize a copy of the adaptor-tagged fragments to link the barcodes. FIGS. 3A and 3B illustrate this method, in which MBC pairing is achieved through circularization of adaptor-tagged fragments. In FIG. 3A, genomic fragments are tagged and amplified (as described in connection with FIG. 1), then converted to single-stranded molecules, such as by denaturing or by treating with lambda exonuclease, generating single-stranded adaptor tagged fragments 302 comprising an insert fragment 304 flanked by a 5′ sequencing tag 306 and a 5′ adaptor 310, and a 3′ sequence tag 308 and a 3′ adaptor 312. In the illustrated embodiment, the 5′ sequencing tag 306 comprises sequence A and a MBC 342, the 3′ sequencing tag 308 comprises sequence B and another MBC 344, the 5′ adaptor 310 comprises adaptor sequence C, and the 3′ adaptor 312 comprises adaptor sequence D; however other arrangements can also be employed. The single-stranded adaptor-tagged fragments 302 are then circularized with the use of a splint oligonucleotide 330. Splint 330 comprises a portion 332 complementary to adaptor sequence D, and a portion 334 complementary to adaptor sequence C. When splint oligonucleotide 330 hybridizes to ends of the adaptor-tagged fragment 302, those ends are brought together, and they may be ligated together by a DNA ligase to form a circularized molecule 336 (shown in FIG. 3B).

In FIG. 3B, the circularized molecule 336 is used to generate a MBC pairing oligo. A portion of the circularized molecule 336 can be amplified using primers 350, 352 which bind to sequences A and B. By amplifying a portion of the circularized molecule 336, linear amplification products 338 can be created which have the two MBCs of the adaptor-tagged in close proximity, allowing for sequencing to determine MBC pairs. In this method, the adaptor-tagged fragments would first be divided into at least two parts; the copies in one part would be used for sequencing the insert fragment and one MBC following mixed-orientation amplification as shown in FIG. 1, and the other portion would be used together with the splint oligo to generate a MBC pairing oligo to be sequenced for barcode linkage.

The splint oligonucleotide can be DNA or RNA. If the splint is RNA, then a ligase may be selected that preferentially ligates two DNA ends put in proximity by an RNA splint, such as SplintR™ Ligase from New England Biolabs. Once the adaptor-tagged fragments are circularized, the reaction can be treated with a DNA exonuclease to remove any remaining non-circularized DNA. A PCR reaction is then done on the circularized products to make copies (i.e., create amplicons of) the region containing the two molecular barcodes and the sequencing primers (FIG. 3B). Sequencing these products give the sequences of the linked molecular barcodes. As an alternative to amplification of the circularized molecule 336, restriction sites 346, 348 can be designed into the ends of the A and B oligos (FIG. 3B), and a linear portion can be cut out of the circular molecule as the MBC pairing oligo and sequenced directly.

Methods for Pairing Insert Sequences Using Known MBC Combinations

In other methods for pairing molecular barcodes on an adaptor-tagged fragment, an MBC pairing oligo is not required to identify MBC pairs. Instead, input fragments are circularized together with a molecule containing a pair of MBCs, hereafter referred to as the circularizing adaptor. A library of circularizing adaptors is used, each member containing a pair of MBC sequences with known combinations-determined by specific design or sequencing measurement. In the illustrated embodiment of FIG. 4, the circularizing adaptor is generated by restriction digestion at sites 410 and 408 of a library of circular DNA molecules 402 containing MBC pairs 406 and 404 in known combinations. The excisable portion 412 is removed, and the resulting circularization adaptor 414 forms a circularized molecule upon ligation to an insert sequence 416. The inserts flanked by MBC pairs can then be amplified for sequencing using primers 418 and 419, generating amplicons 420. An exonuclease can optionally be utilized to remove non-circularized DNA fragments prior to amplification. The circularizing adaptor can be prepared by any suitable method which produces a pair of MBC sequences adjacent to ligatable ends. For example, oligo libraries containing known MBC pairs can be synthesized and inserted into a linearized vector by ligation to form the pre-adaptor structure 402 in FIG. 4. Alternatively, one or multiple fragments containing randomized MBCs can be inserted, with the MBC pairing measured by sequencing a portion of the pre-adaptor pool. Still other embodiments of this approach involve combining synthesized MBC-containing oligo libraries into pre-defined pairs based on complementary base pairing. For the approaches described above (FIGS. 2-4), pairing of single-end reads can be done in silico on the basis of the MBC sequences. For approaches involving the pairing oligo (FIGS. 2-3), the pairing oligos can be sequenced either together or separately from the insert library. If two MBC sequences are observed linked on a pairing oligo read and those same sequences are observed on MBC reads linked to two insert sequences, those inserts are candidate pairs. Higher pairing confidence can be obtained through proximal alignment position of the insert, overlapping insert sequences, and the use of longer MBCs to decrease the likelihood of multiple inserts having the same MBC sequence. For the approach utilizing a known pair of MBCs, a similar technique is employed to pair single-end insert reads, except the MBC pairing is known separately from the insert sequencing without the need for a pairing oligo.

Methods for Pairing Insert Sequences with One Randomized MBC

As another aspect, the present disclosure describes novel methods for pairing single-end sequencing reads from adaptor-tagged fragments having a single MBC.

As for the approaches described above the present methods comprise introducing adaptor-tagged fragments having inverted insert sequences into a sequencing system. The inverted adaptor-tagged fragments can be prepared as described in FIG. 1. In contrast to the previous methods which identify pairs of reads for an insert on the basis of two linked MBCs, in some embodiments the present methods identify pairs by linking reads with complementary sequences of one MBC. This can be done by sequencing amplicons comprising both orientations of an insert together with its MBC. The MBC sequences can be determined for each orientation either by conducting separate insert and barcode sequencing reads, or alternatively by sequencing through the insert from one end to the other. If there are no errors introduced in the MBC sequence, the MBC sequence from one orientation will be the reverse complement of the MBC sequence from the second orientation. In one embodiment, the adaptor-tagged fragments with both orientations of adaptors are sequenced simultaneously by duplexing primers for reading the fragment sequence, and separately duplexing primers for reading the barcode. In another embodiment, the forward or A orientation may be sequenced in one sequencing run, and the inverted or B orientation may be sequenced in a different sequencing run. In another embodiment, different sequencing runs may comprise different combinations of different orientations (for example, the mixed library may comprise 90% of the forward or A orientation and 10% of the inverted or B orientation), depending on how much pairing was required. As a result, sequence reads will be generated from both ends and from both strands of an input fragment and can be linked together through a shared or complementary molecular barcode (or through linked molecular barcodes at each end).

FIGS. 5A and 5B illustrates an embodiment of the present methods, in which a library is generated with two orientations of adaptors relative to the sequence of the input fragment. In FIG. 5A, tagged fragments are prepared by attaching sequence tags 506, 508 to input fragment 504. Sequence tag 508 comprises sequence B, and sequence tag 506 comprises a molecular-barcode-containing sequence A, which has subsequences A1, N, and A2. Tagged fragment 502 is amplified by PCR using primers 507, 509 which bind sequences A1 and B. In FIG. 5B, copies of tagged fragment 502 are further amplified with primers 510 and 516 to attach sequence adaptors C and D in two orientations: with C attached to sequence tag A and D to sequence tag B (Orientation A) and the reciprocal with primers 512 and 514 (Orientation B). Adaptor-tagged fragments 520, 522 from this PCR are pooled and sequenced.

FIGS. 6A and 6B illustrate how a library of adaptor-tagged fragments can be sequenced following cluster generation on a solid surface of a sequencing system. FIG. 6A illustrates the duplexing of sequencing primers for obtaining sequence reads of the fragment, and both strands of the MBC. Adaptor-tagged formats 520 and 522 from FIG. 5B have been loaded on the solid support 601 (e.g., a flow cell) of a sequencing system. Clusters 602, 604 comprising identical copies of fragments 520, 522 have been generated. Specifically, Read 1 of orientation A will be primed with primer 610 (Primer A2), and will start the insert sequencing read with the insert sequence G1 (read off of the template corresponding to G1′, the complement to G1). Subsequently in cluster 602, the molecular barcode will be primed with the primer 612 (primer A1), and will have the sequence N (read off of the template corresponding to N′, the complement to N). Meanwhile, on the same flow cell, other clusters (such as cluster 604) will be generated from the same input fragment but will be in the B Orientation. Here, Read 1 of orientation B will be primed with primer 614 (Primer B′), and will start the fragment sequencing read with the fragment sequence G2′ (which is read off of the template corresponding to G2, the complement to G2′). Subsequently in this B cluster, the molecular barcode or index sequence will be primed with the primer A2′, and will have the sequence N′ (read off of the template corresponding to N, the complement to N′.) In FIG. 6A, a proportion of adaptor-tagged fragments in the library will generate clusters with both orientations A and B. Sequencing ‘read 1’ using indicated read 1 primers A2 and B′ will generate genomic sequence from opposite ends of the fragment (G1 and G2). A separate barcode read using primers A1 and A2′ will generate complementary barcode sequences. FIG. 6B illustrates that genomic sequences originating from opposite ends of the same fragment can be linked in silico through their complementary index sequences, enabling a sequence determination of longer length than the sequencing read.

Therefore, as shown in FIG. 6B, a total of 4 sequences can be generated from the A and B orientation generated from the original, barcoded input fragment: Sequence reads 620 (G1) and sequence 622 (G2′) corresponding to the ends of the input fragment, and sequence reads 624 and 626 (N and N′) corresponding to the sequence and the reverse complement of the barcode on the adaptor-tagged fragment. Sequence reads 620 and 622 can be aligned to provide sequence information 628 having a longer length than the individual reads.

Pairing of insert reads is determined by complementary MBC sequences. As for the methods described above, pairing confidence can be increased through overlapping insert sequence, proximal insert alignment positions, and longer MBC sequences. When only one of the sequence tags comprises an MBC, it may be desirable for the molecular barcode sequences to be long enough or unique enough to link the G1 and G2 sequences with little ambiguity. For example, a 8-nt molecular barcode consisting of random “N” nucleotides would correspond to approximately 65,000 different sequences (or 32,000 pairs of sequences with their reverse complements). In some cases, where there are many millions of sequencing reads to pair, there could be ambiguity as to whether a given sequence AATTGC is a unique sequence for orientation A, or the complement of the barcode GCAATT in orientation B. This ambiguity would be further increased by considering possible sequencing or amplification errors in the molecular barcodes (such as whether ATTTGC is related to AATTGC, or unique.) However, this potential ambiguity can be addressed by using longer molecular barcodes, or by combining the information from the barcode sequence(s) with information from the insert sequence(s). For example, a 16-nt molecular barcode of random N nucleotide would correspond to over 4 billion sequences (or 2 billion pairs of sequences with their reverse complements), making it likely that each barcode sequence and its complement would only occur once or a few times in a sequencing experiment with less than a billion reads. In this case, the barcode N and the reverse complement N′ could be more confidently paired to link insert reads G1 and G2′ to lengthen the alignment and/or for error reduction. Thus, sequence reads from opposite ends of the input fragment can be combined into a sequence determination of potentially longer length than the sequencing read.

In some embodiments, the barcodes may contain structure and/or information in addition to providing a stretch of random nucleotides. For example, rather than having an MBC have the sequence NNNNNNNN paired to N′N′N′N′N′N′N′N′, asymmetrical barcodes could be used, such as YNNNNNNY, where Y corresponds to C or T (or, G or A). In this case, the total diversity of the barcode sequences would go down, but the orientation would be encoded. In this example, when a MBC sequence of CGATTCTT is obtained, it is known to indicate one orientation (e.g., orientation A) while AAGAATCG would be the complementary barcode, and the presence of A and G in this barcode sequence also indicates it must be from orientation B. In another example, a random or semi-random MBC (e.g., with thousands, millions, or billions of combinations) could be combined with a sample index barcode of a more limited sequence (e.g., with 4, 8, 16, 96, or 384 known combinations). For instance, a barcode could have the structure NNNNiiiiiiNNNN, where N represent degenerate bases as a molecular barcode and i bases represent a defined sequence assigned to a particular sample. In this way, a sample index portion of the barcode can also be used to define the read orientation, as long as non-complementary sample indices are chosen. In other embodiments, a complex but non-random set of MBCs could be used, and these sequences could be designed such that the list of MBCs and their complements do not overlap with the sequences of sample indices used in the sequencing experiment, or their complements.

In many cases, the sequence information from the input fragment itself can add useful information that would help in pairing sequence reads from the A and B orientations. In cases where the ends of the input fragments are generated by a random process such as shearing, the start-site and end-site of an input fragment may be different from many, or even all, other input fragments in the library. This sequence information could be used in conjunction with the barcode information to increase the confidence of pairing, or for error correction of either the fragment read or the barcode read. For example, if there is an input fragment with a 200 base sequence and Read 1 from orientations A and B are each 120 nucleotides, the reads from that fragment should be on opposite strands, with start sites 200 bp apart, and an overlap region of 40 bp in the middle. In this case, the pairing of the two reads from the orientations would enable error-correction in the overlapped region. Use of input fragments generally smaller than the read length would enable full overlap of the insert sequences, and would also supply both start-site and end-site information in each orientation. In some embodiments where higher confidence is desired or where the sequencing platform has a high intrinsic error rate, the fragment size and sequencing read length may be chosen to maximize the overlapped region. Even in cases where the length of the input fragment is longer than 2× the read length, and there is no overlapped region, the genomic coordinates of the reads can be used to increase the confidence of pairing: reads from the same input fragment should be mapped to both strands, the start sites should be a predictable distance apart (typically sequencing libraries would have fragments less than 1 kb, less than 500 bp, less than 300 bp, or in the case of FFPE samples, may be less than 150 bp). Therefore, a sequencing read on the (+) strand is likely to be paired with a read on the (−) strand that is 250 bp away, but it would not be paired with a read on the (+) strand that is 250 bp away, or a read on the (−) strand that is 2.5 kb away. In some embodiments it may be advantageous to use only a narrow size range of fragments (e.g., 250-300 bp), to increase the confidence of pairing. In other embodiments, a wider size range may be used, or a mixture of size ranges (e.g., one population of 250 bp fragments could be combined with a second population of 800 bp or 1 kb fragments.)

The skilled artisan would recognize in light of the present disclosure that there are many possible ways in which to use non-random combinations of barcode and sample index sequences, or combinations of barcodes and information from the insert sequences, to increase the confidence of pairing the reads from both ends of the input fragment. For example, non-random MBCs may be designed or combined with known sequences to identify errors such as insertions or deletions in the MBC sequence. For example, longer MBC's may be used to decrease pairing ambiguity in applications with less input fragment complexity, such as multiplex amplicon sequencing, where the start-site and stop-sites of the fragments are determined by the original PCR primers.

In some embodiments, the locations of the molecular barcode, sample index, and primer sequences could be changed, or different forms of adapter may be used. For example, the present methods could be used with Y-shaped adapters described in Gormley et al. US Pat. App. Pub. No. 20070128624, or with loop-shaped adapters as described in Hendrickson US Pat. App. Pub. No. 20120238738. Following the teachings of the present disclosure, one can design appropriate sets of amplification primers and sequencing primers, to enable the amplification and sequencing of the input fragment in two orientations.

In some embodiments, the sequencing primers or sequencing protocol could be designed to sequence a short stretch of the adapter oligonucleotide (for example, 1 to 3 bases), before or after sequencing the barcode or insert sequence. If the adapters are designed to have orientation-specific sequences in these regions, this would have the advantage of enabling decoding of the orientation of the cluster, independently from the sequence. For example, in FIG. 6A, if the A2 and B′ primers were shortened such that they sequenced two bases of the A2′ adapter and B adapters, respectively, this would allow the user to know which orientation each cluster is in. A similar result could be obtained by sequencing past the length of the input fragment or barcode region, and into the adapter sequence itself. Alternatively, the primers specific for the two orientations could be labeled with a cleavable fluorescent dye, or fluorescent probes specific for the two orientations could be hybridized, scanned, and removed before sequencing. The advantage of these embodiments is that it may give higher confidence for pairing the molecular barcodes with their reverse complements. For example, a barcode such as AACC″ may either be paired with GGTT, or they could be independent barcodes in the same orientation; whereas a barcode AACC (from Orientation A) may be paired more confidently with GGTT (from Orientation B).

The present methods provide several advantages over conventional paired-end reads. The present methods are not limited to sequencing systems from a specific vendor such as Illumina, as is currently the case for paired-end sequencing. For example, virtual pairing of sequence reads could be used for a nanopore sequencing platform, where pairing of reads from the + and − strands of the same template could be used for error correction. In cases of sequencing platforms with longer reads and/or higher error rates, it may be desirable to use significantly longer MBC and/or insert sequences, to increase the confidence of pairing and make the method more robust to sequencing errors. An additional benefit over paired-end sequencing is that both ends of the genomic fragments can be sequenced simultaneously. In contrast, paired-end sequencing relies on sequential sequencing of the two strands, and thus increases the time required for the sequencing experiment, compared to single-end sequencing. An advantage over synthetic long read technology is that no dedicated equipment (e.g. droplet generator) is required for this approach. Moreover, lower read depth is needed since only two reads are linked, versus many for synthetic long reads. An advantage over dedicated approaches such as circularizing long genomic fragments is that the present methods integrate smoothly into a library preparation procedure for a typical sequencing application such as clinical sequencing, with minimal procedural changes. Furthermore, the utility of the sequence data for detecting common aberrations of interest such as SNVs or CNVs is not compromised, unlike a dedicated method such as employing circularization of long fragments.

Another advantage of the present methods is that they can be implemented in many different ways and yield meaningful results. For example, input fragments having two different orientations relative to an adaptor may either be pooled and sequenced simultaneously in the same sequencing run, or they could be sequenced separately, in different runs or in different flow cell lanes (or different locations on a solid support). An advantage of sequencing the orientations separately is that the user may gain useful information from the first run: for example, if the sequencing read depth of orientation A is too high or too low, this could be adjusted before sequencing orientation B (or before sequencing a mixture of orientations A and B, which would not need to be a 50-50 mix.) Also, sequencing the different orientations separately would remove any ambiguity of the orientation of the input fragment and the barcode region, which may help in pairing. The present methods also make it possible to seed a sequencing system (such as a flow cell) with both orientations, but to selectively sequence only the fraction of clusters in one orientation, using only one of the sequencing primers. This could be useful in cases where cluster density would otherwise be too high; the sequencing data from the two orientations could be collected sequentially from the same flow cell, rather than simultaneously. In some embodiments, this could be used as an advantage, in that sequential sequencing runs could be used to substantially increase the amount of sequence data provided from a single flow cell.

Aligning Sequence Reads from Inverted Input Fragments

In some embodiments, the present methods comprise aligning sequence reads of the adaptor-tagged fragments. The sequence reads may be processed and grouped in any suitable way. In some embodiments, the sequence reads may be initially grouped by the fragment sequence and/or the barcode(s). In some implementations, initial processing of the sequence reads may include identification of molecular barcodes (including sample identifier sequences or sub-sample identifier sequences), and/or trimming reads to remove low quality or adaptor sequences. In addition, quality assessment metrics can be run to ensure that the dataset is of an acceptable quality. In some embodiments therefore, the method may comprise identifying identical or near-identical sequence reads that have identical or near-identical fragmentation breakpoints but different primer sequences and/or barcode sequences. As would be apparent, the confidence that a potential sequence variation is a true variation (rather than a PCR or sequencing error) increases if it is present in more than one molecule. Likewise, copy number variations can be measured more accurately if one can distinguish fragments that are otherwise identical to one another.

In some embodiments, a sequencing run or sequencing experiment may produce at least 100, at least 1,000, at least 10,000, at least 1,000,000, up to 100,000,000,000 or more sequence reads. The length of the sequence reads may vary depending on, for example, the platform used. In some embodiments, the length of sequence reads may be in the region of 30 to 800 bases.

Sequence reads can be assembled to obtain a plurality of discrete sequence assemblies that each corresponds to a potential input fragment sequence. Sequence reads may be assembled using any suitable method. In some embodiments, sequence reads can be assembled by aligning each read to a reference sequence, such as a reference genome. In some embodiments, at least one assembled sequence obtained from the sequence reads aligns to a reference sequence. Such alignment can be done manually or by a computer algorithm, such as a Burrows-Wheeler Aligner (BWA), or the Efficient Local Alignment of Nucleotide Data (ELAND) computer program distributed as part of the Illumina Genomics Analysts pipeline. The matching of a sequence read in aligning can be a 100% sequence match or less than 100% (non-perfect match). In some embodiments, MBC sequences may be used to group sequences or identify different orientations prior to alignment of the sequences to a reference.

In some embodiments, graph theory is used to assemble the reads. In particular cases, assembling the sequence reads may comprise making a directed graph, such as a de Bruijn graph. The use of de-Bruijn graphs to assemble reads is described in U.S. Pat. No. 8,209,130; U.S. Pub. 2011/0004413, U.S. Pub. 2011/0015863, and U.S. Pub. 2010/0063742, which are incorporated by reference herein.

### Kits for Making a Library of Inverted Input Fragments

As another aspect of the present invention, kits are provided which comprise primer sets for making adaptor-tagged fragments as described herein. In addition to above-mentioned components, the kits may further include instructions for using the components of the kit to practice the present methods, i.e., to instructions for sample analysis. The instructions for practicing the present methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, portable drive, or cloud-based storage, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

### Example 1

In this example, an experiment was conducted to test an embodiment of the present sequencing methods. A library was prepared by enriching a polynucleotide sample using Agilent's ClearSeq Cancer Panel. 10 ng of DNA harboring a known translocation between EML4 and ALK, at 50% allele frequency, was used. The library was prepared according to the Agilent XTHS library preparation kit and SureSelect protocol, following the manufacturer's instructions. The sequences of oligos used for this example are given in Table 1 below. Briefly, genomic DNA was sheared by sonication, repaired, adenylated, and ligated to a mixture of ‘A’ and ‘B’ duplex adaptors comprising a single thymine 3′ overhang. The ‘A’ adaptor contained 3 regions: A1, N, and A2 as described above, with the N region comprising a 10-base randomized MBC and a 4 base sample index; the B adaptor contained only one region and no MBC. The resulting fragments were amplified with primers complementary to A1 and B, followed by target enrichment using Agilent Technologies ClearSeq Comprehensive Cancer panel. Captured amplicons were then subjected to a first stage of post-enrichment PCR with the same primers A1′ and B′. Subsequently, modifications from the standard procedure were then introduced to mixed orientation amplicons; the product of the first stage post-enrichment PCR was split, and two further amplifications were carried out to add sequence adaptors in two orientations, as illustrated in FIG. 5B. The resulting products were pooled and sequenced on an Illumina MiSeq, duplexing insert and barcode sequencing primers. For data analysis, insert reads were considered paired based on one of two qualifications: ‘proximal’ read pairs were linked by complementary MBC sequences and an alignment position within 1 kilobase on the human genome. Alternatively, ‘distal’ read pairs, useful for identifying translocations or other genomic rearrangements, were identified by complementary MBC sequences as well as alignments to positions linked by at least five unique MBCs.

The results of this experiment (summarized in Table 2) demonstrate that a substantial proportion of the sequence reads can be paired by this approach. One advantage demonstrated in this example is the identification of the EML4-ALK gene fusion. No single reads resulted in alignments to both gene fusion partners, underscoring the challenge of identifying translocations from single-end sequencing reads. However, the virtual read pairing of this disclosure enabled detection of the translocation by linking multiple reads derived from opposite ends of fragments covering the translocation break points.

TABLE 1

| Oligo Name | Oligo Sequence |
|---|---|
| AdapAFor | GCAATCGTCGATAGCGTTGNNNNNNNNNNTTCCTGC |
| | TCGTGCGACGCATAACCGTTCGATCGTTACT |
| AdapARev | GTAACGATCGAAddC |
| AdapBFor | TATGACGCGTCGTATGCT |
| AdapBRev | GCATACGACGCGTddC |
| PCR1_for | ACGTACCGTTCGCAATCGTCGATAGCGTTG |
| PCR1_rev | TATGACGCGTCGTATGCT |
| PCR2_Afor | AATGATACGGCGACCACCGAGATCTACACACGTACC |
| | GTTCGCAATCGTCGATAGCGTTG |
| PCR2_Arev | CAAGCAGAAGACGGCATACGAGATTATGACGCGTCGT |
| | ATGCT |
| PCR2_Bfor | CAAGCAGAAGACGGCATACGAGATACGTACCGTTCGC |
| | AATCGTCGATAGCGTTG |
| PCR2_Brev | AATGATACGGCGACCACCGAGATCTACACCGACAGGT |
| | TCAGTATGACGCGTCGTATGCT |
| InsertReadA | CGTGCGACGCATAACCGTTCGATCGTTACT |
| InsertReadB | CGACAGGTTCAGTATGACGCGTCGTATGCT |
| IndexReadA | CACGTACCGTTCGCAATCGTCGATAGCGTTG |
| IndexReadB | AGTAACGATCGAACGGTTATGCGTCGCACG |

TABLE 2

| | A orientation | A + B |
|---|---|---|
| | only | orientation |
| number of ‘A’ reads | 11,380,012 | 4,913,063 |
| number of ‘B’ reads | n/a | 6,466,949 |
| Percent of distinct ‘A’ reads matched to a ‘B’ | n/a | 46% |
| pair | | |
| # alignments within 1 Kb of translocation | 17,044 | 16,755 |
| junction | | |
| # paired alignments within 1 Kb of | n/a | 4,586 |
| translocation junction | | |
| # of reads with alignments to both EML4 and | 0 | 0 |
| Alk | | |
| # of paired alignments linking EML4 and Alk | n/a | 45 |

Multiple barcodes that support linking of sequence reads to a single input fragment, despite being sequences from distant genomic regions (based on the reference genome), enable identification of genomic translocation with high statistical confidence. The rate of spurious false pairings determines the minimal number of independent events necessary to support the calling of a putative translocation event. In this experiment, 11 distinct barcodes linked the fusion of EML4 and ALK genes.

### Exemplary Embodiments

Embodiment 1. A method of pairing sequencing reads generated from a library of nucleic acids comprising: ligating one or more sequence tags to each end of an input fragment to produce a tagged fragment, wherein the input fragment comprises an insert sequence, wherein at least one of said sequence tags comprises a molecular barcode, performing a first-stage amplification of the tagged fragment with primers complementary to the sequence tags to produce a plurality of double-stranded amplicons comprising the insert sequence; performing a second-stage amplification with two or more primers which anneal to at least part of the sequence tags and add sequencing adaptor sequences in such a way as to generate a library of amplicons comprising the insert sequence in at least two different orientations with respect to the sequencing adaptors; sequencing said library on a next-generation sequencing platform in such a way as to obtain sequence reads for the insert and the molecular barcode sequences; and using the molecular barcode reads to identify pairs of reads of the insert sequences derived from the same input fragment and sequenced from the different orientations.

Embodiment 2. The method of embodiment 1, where one molecular barcode is attached to the input fragment, and pairs of reads of the insert sequence are identified at least partially on the basis of complementary molecular barcode reads.

Embodiment 3. The method of embodiment 2, where the molecular barcode sequencing read contains sequences which impart information regarding the insert orientation.

Embodiment 4. The method of any of embodiments 1 to 3, where two molecular barcodes are attached to each input fragment.

Embodiment 5. The method of embodiment 4, further comprising generating a pairing oligo to identify combinations of molecular barcodes attached to an input fragment to be used in pairing single-end reads.

Embodiment 6. The method of embodiment 5, where a pairing oligo shorter than the input fragment is generated by annealing two oligos, wherein one of the oligos has regions complementary to both ends of the first-stage amplification products, followed by extension and ligation.

Embodiment 7. The method of embodiment 5, where a pairing oligo is generated by annealing each end of a tagged fragment to a splint oligonucleotide, ligating to form a circularized fragment, and amplifying a region of the circularized fragment containing the two molecular barcode sequences.

Embodiment 8. The method of embodiment 7, wherein the splint oligonucleotide is a DNA oligonucleotide.

Embodiment 9. The method of embodiment 7, wherein the splint oligonucleotide is an RNA oligonucleotide.

Embodiment 10. The method of embodiment 7, further comprising an exonuclease step to remove non-circularized DNA.

Embodiment 11. The method of embodiment 7, wherein sequence tags contain restriction sites adapted for generating the pairing oligo following circularization of the tagged fragments.

Embodiment 12. The method of embodiment 4, where the combinations of molecular barcodes are designated on the basis of a circularizing adaptor.

Embodiment 13. The method of embodiment 12, where the circularizing adaptor is generated by restriction digestion Embodiment f a circularized molecule containing two molecular barcodes.

Embodiment 14. The method of embodiment 13, where the two molecular barcodes are designed and synthesized as an oligo library prior to integration into a circularized vector.

Embodiment 15. The method of embodiment 13, where the two molecular barcodes are randomized molecular barcodes, and the combination of the randomized MBCs is determined by sequencing the region of the circularized vector containing the molecular barcodes separately from the sequencing of the inserts.

Embodiment 16. The method of embodiment 12, where the circularization adaptor is generated by annealing two oligo libraries containing designed molecular barcodes on the basis of complementary base pairing.

Embodiment 17. The method of any of embodiments 1 to 16, where the two orientations of the insert sequence are sequenced simultaneously.

Embodiment 18. The method of any of embodiments 1 to 16, where the two orientations of the insert sequence are sequenced in separate sequencing runs.

Embodiment 19. The method of any of embodiments 1 to 18, where the insert and molecular barcode sequences are determined by sequential sequencing reads.

Embodiment 20. The method of any of embodiments 1 to 18, where the insert and molecular barcode sequences are determined by a single sequencing read.

Embodiment 21. The method of embodiment 17, where the two fragment orientations are sequenced using different sequencing primers for the different orientations.

Embodiment 22. The method of embodiment 21, where the two insert orientations are sequenced using 2 different sequencing primers for the different orientations, and the barcodes are sequenced using 2 different barcode sequencing primers.

Embodiment 23. The method of embodiment 21, where the two fragment orientations are sequenced in separate clusters or beads, using different sequencing primers for the different orientations.

Embodiment 24. The method of any of embodiments 1 to 23, further comprising using sequence information from the inserts, such as genomic coordinates, start-site or end-sites, or overlapping regions of the inserts, to determine the sequence read pairs.

Embodiment 25. The method of claim 2, further comprising using sequence information from the inserts, such as genomic coordinates, start-site or end-sites, or overlapping regions of the inserts, to determine the sequence read pairs.

Embodiment 26. A method of making a sequencing library of nucleic acids comprising: attaching first sequence tag to at least one end of an input fragment comprising an insert sequence to produce a tagged fragment, wherein the first sequence tag comprises sequence A; amplifying the tagged fragment to produce a plurality of tagged fragments comprising the insert sequence, and at least some of the tagged fragments comprise a strand comprising a 5′ sequence tag comprising sequence A, wherein sequence A comprises a primer binding site; amplifying the top strand of the tagged fragments with a primer set comprising primers of formulas C-A, and D-A to produce adaptor-tagged fragments, wherein sequences C and D are adaptor sequences; wherein a first set of the adaptor-tagged fragments comprise a strand comprising 5′-end comprising sequences C and A, and the insert sequence; and wherein a second set of the adaptor-tagged fragments comprises a strand comprising a 5′ end comprising sequences D and A, and the insert sequence.

Embodiment 27. The method of embodiment 26, wherein the input fragment sequence in the first set is inverted compared to the input fragment sequence in the second set, relative to an adaptor sequence common to both the first and second sets of adaptor-tagged fragments.

Embodiment 28. The method of any of embodiments 26 or 27, wherein either the first sequence tag or the second sequence tag comprises a molecular barcode.

Embodiment 29. The method of embodiment 28, wherein the first sequence tag has formula A1-N-A2, wherein N is a barcode sequence, and A1 and A2 are primer binding sites.

Embodiment 30. The method of embodiment 28, wherein the library comprises adaptor-tagged fragments of formulas C-A-G-B-D and D-A-G-B-C, where G has a sequence of the input fragment.

Embodiment 31. The method of any of embodiments 26 to 30, wherein one or both of the first and second sequence tags comprises an asymmetrical barcode of formula YNNNNNNY, wherein N is A, C, T, or G, and Y is C or T.

Embodiment 32. The method of any of embodiments 26 to 30, wherein the first and second sequence tags both comprise molecular barcodes (MBC).

Embodiment 33. The method of embodiment 32, further comprising generating an MBC pairing oligonucleotide from the adaptor-tagged fragment.

Embodiment 34. The method of embodiment 33, wherein the MBC pairing oligo is generated by: annealing first and second pairing primers to the adaptor-tagged fragment wherein the first pairing primer anneals to sequence D, and the second pairing primer anneals to both A and B; and ligating the extended pairing primers to produce the molecular barcode pairing oligonucleotide.

Embodiment 35. The method of embodiment 34, wherein the pairing primers are sequentially annealed to and extended along the adaptor-tagged fragment.

Embodiment 36. The method of embodiment 34, wherein the pairing primers are substantially simultaneous annealed and extended.

Embodiment 37. The method of embodiment 33, wherein the molecular barcode pairing oligonucleotide is sequenced in a sequencing run with the adaptor-tagged fragments.

Embodiment 38. The method of embodiment 37, wherein the analysis of sequencing data comprises determining sequences of each MBC in the molecular barcode pairing oligonucleotides to identify MBC pairs, and using the MBC pairs to identify pairs of sequence reads from different orientations of the input fragment.

Embodiment 39. The method of embodiment 33, wherein the MBC pairing oligo is generated by: circularizing an adaptor-tagged fragment by hybridization to a splint oligonucleotide, wherein the splint has formula C-D or D′-C′ to link the molecular barcodes; ligating the ends of the adaptor-tagged fragment to generate a circularized adaptor-tagged fragment; and amplifying a region of the circularized fragment comprising the molecular barcodes with primers that bind sequences A and B, or complements thereof, to produce the molecular barcode pairing oligonucleotide.

Embodiment 40. The method of embodiment 39, wherein the splint oligonucleotide is a DNA oligonucleotide.

Embodiment 41. The method of embodiment 39, wherein the splint oligonucleotide is an RNA oligonucleotide.

Embodiment 42. The method of embodiment 39, further comprising an exonuclease step to remove non-circularized DNA.

Embodiment 43. The method of embodiment 39, wherein sequences A and B comprise restriction sites, and the method further comprises cutting the circularized fragments with a restriction enzyme to produce the MBC pairing oligo.

Embodiment 44. The method of any of embodiments 26 to 43, wherein the first and second sequence tags are attached to the ends of the polynucleotide fragments by ligating the polynucleotide fragments into a vector comprising predetermined pairs of molecular barcodes.

Embodiment 45. The method of any of embodiments 26 to 44, wherein sequences C and D are capture sequences configured for a solid support of a sequencing system.

Embodiment 46. The method of embodiment 45, wherein the library is loaded onto a flow cell comprising binding sites for one or more of sequences C, C′, D, or D′.

Embodiment 47. The method of embodiment 45, wherein the library is loaded onto capture beads comprising binding sites for one or more of sequences C, C′, D, or D′.

Embodiment 48. The method of any of embodiments 26 to 47, wherein the input fragments are genomic DNA fragments or cDNA fragments.

Embodiment 49. The method of any of embodiments 26 to 48, further comprising sequencing the library by primer extension with a sequencing primer set so that both strands of the input fragments are sequenced simultaneously to produce sequencing reads from both ends of the input fragments, analyzing sequencing data such that sequence reads from both ends of the input fragment can be paired, thereby generating a sequencing determination for the input fragment having greater length than the sequence reads from a single sequencing run.

Embodiment 50. A method of sequencing a library comprises adaptor-tagged fragments, the method comprising: introducing first and second sets of the adaptor-tagged fragments to a solid support of a sequencing system, wherein the first set comprises adaptor-tagged fragments of formula C-A-G-B-D and/or a complement thereof, and the second comprises adaptor-tagged fragments of formula D-A-G-B-C and/or a complement thereof, wherein sequences A and B comprise primer binding sites and molecular barcodes, sequences C and D are adaptor sequences, and G comprises a sequence of an input fragment, and wherein the solid support comprising binding sites for one or more of sequences C, C′, D, and D′. The method also comprises introducing a first set of sequencing primers to the solid support, wherein the first set comprises (a) sequencing primers that bind to sequence A and sequencing primers that bind to sequence B′, or (b) sequencing primers that bind to sequence A′ and sequencing primers that bind to sequence B; sequencing the fragment sequences of the first and second sets of the adaptor-tagged fragments to obtain sequence reads from different orientations of the insert sequence simultaneously; introducing a second set of sequencing primers which bind to regions downstream of (3′ to) the MBC; determining complementary sequences of the molecular barcodes from different orientations of the adaptor-tagged fragments simultaneously; and analyzing the sequencing data to pair sequencing reads from different orientations of one of the insert sequences.

Embodiment 51. The method of embodiment 50, wherein the sequencing data comprises: sequence reads for at least two portions of one of the insert sequences, wherein each of the portions are at opposite ends of the input fragment; and sequence reads for one or more molecular barcodes attached to the fragment.

Embodiment 52. A method of sequencing a library of adaptor-tagged fragments comprising: introducing the library to a solid support of a sequencing system, wherein the library comprises: a first set of adaptor-tagged fragments wherein a strand has formula C-A1-N-A2-G-B-D, or its complement, and a second set of adaptor-tagged fragments wherein a strand has formula D-A1-N-A2-G-B-C, or its complement, wherein sequences A1, A2 and B are primer binding sites, N is a barcode, sequences C and D are capture sites for a sequencing system, and sequence G is a sequence of the input fragment, and the solid support comprising binding sites for one or more of sequences C, C′, D, and D′. The method also comprises obtaining sequence reads from both ends of sequence G by introducing a set of sequencing primers to the solid support, wherein the set comprises (a) a sequencing primer that binds to sequence B and a sequencing primer that binds to sequence A2′, or (b) a sequencing primer that binds to sequence B′ and a sequencing primer that binds to sequence A2, and by extending the sequencing primers to produce sequencing data. The method also comprises obtaining sequence reads from both ends of N by introducing a set of sequencing primers to the solid support, wherein the set comprises (a) sequencing primers that bind to sequence A1 and sequencing primers that bind to sequence A2′, or (b) sequencing primers that bind to sequence A1′ and sequencing primers that bind to sequence A2, and extending the sequencing primers to produce sequencing data. The method also comprises analyzing the sequence reads for sequence G and sequence N and pairing sequence reads for both ends of sequence G to generate a sequence determination for sequence G longer than the sequence reads.

Embodiment 53. The method of embodiment 52, wherein sequence G is sequenced from different orientations simultaneously.

Embodiment 54. The method of any of embodiments 52 or 53, wherein sequence N is sequenced from different orientations simultaneously.

Embodiment 55. The method of any of embodiments 52 to 54, further comprising analyzing the sequencing data to pair sequencing reads from different orientations of the input fragments.

Embodiment 56. The method of any of embodiments 52 to 55, wherein sequence N has a formula NNNNNNNN, wherein each N is A, C, T or G.

Embodiment 57. The method of any of embodiments 52 to 55, wherein sequence N has a formula YNNNNNNY, wherein each N is A, C, T or G, and Y is C or T, or G and A.

Embodiment 58. The method of any of embodiments 52 to 57, wherein sequence M has a formula NNNNiiiiiiNNNN, where N represent degenerate bases as a molecular barcode and i represents a defined sequence.

Embodiment 59. The method of any of embodiments 26 to 58, further comprising analyzing sequence information from the input fragment to generate the sequence determination.

In view of this disclosure it is noted that the methods and kits can be implemented in keeping with the present teachings. Further, the various components, materials, structures and parameters are included by way of illustration and example only and not in any limiting sense. In view of this disclosure, the present teachings can be implemented in other applications and components, materials, structures and equipment to implement these applications can be determined, while remaining within the scope of the appended claims.

1. A method of pairing sequencing reads generated from a library of nucleic acids comprising:
ligating one or more sequence tags to each end of an input fragment to produce a tagged fragment, wherein the input fragment comprises an insert sequence, wherein at least one of said sequence tags comprises a molecular barcode,
performing a first-stage amplification of the tagged fragment with primers complementary to the sequence tags to produce a plurality of double-stranded amplicons comprising the insert sequence;
performing a second-stage amplification with two or more primers which anneal to at least part of the sequence tags and add sequencing adaptor sequences in such a way as to generate a library of amplicons comprising the insert sequence in at least two different orientations with respect to the sequencing adaptors;
sequencing said library on a next-generation sequencing platform in such a way as to obtain sequence reads for the insert and the molecular barcode sequences; and
using the molecular barcode reads to identify pairs of reads of the insert sequences derived from the same input fragment and sequenced from the different orientations.

2. The method of claim 1, where one molecular barcode is attached to the input fragment, and pairs of reads of the insert sequence are identified at least partially on the basis of complementary molecular barcode reads.

3. The method of claim 2, where the molecular barcode sequencing read contains sequences which impart information regarding the insert orientation.

4. The method of claim 1, where two molecular barcodes are attached to each input fragment.

5. The method of claim 4, further comprising generating a pairing oligo to identify combinations of molecular barcodes attached to an input fragment to be used in pairing single-end reads.

6. The method of claim 5, where a pairing oligo shorter than the input fragment is generated by annealing two oligos, wherein one of the oligos has regions complementary to both ends of the first-stage amplification products, followed by extension and ligation.

7. The method of claim 5, where a pairing oligo is generated by annealing each end of a tagged fragment to a splint oligonucleotide, ligating to form a circularized fragment, and amplifying a region of the circularized fragment containing the two molecular barcode sequences.

8. The method of claim 7, wherein the splint oligonucleotide is a DNA oligonucleotide.

9. The method of claim 7, wherein the splint oligonucleotide is an RNA oligonucleotide.

10. The method of claim 7, further comprising an exonuclease step to remove non-circularized DNA.

11. The method of claim 7, wherein sequence tags contain restriction sites adapted for generating the pairing oligo following circularization of the tagged fragments.

12. The method of claim 4, where the combinations of molecular barcodes are designated on the basis of a circularizing adaptor.

13. The method of claim 12, where the circularizing adaptor is generated by restriction digestion of a circularized molecule containing two molecular barcodes.

14. The method of claim 13, where the two molecular barcodes are designed and synthesized as an oligo library prior to integration into a circularized vector.

15. The method of claim 13, where the two molecular barcodes are randomized molecular barcodes, and the combination of the randomized MBCs is determined by sequencing the region of the circularized vector containing the molecular barcodes separately from the sequencing of the inserts.

16. The method of claim 12, where the circularization adaptor is generated by annealing two oligo libraries containing designed molecular barcodes on the basis of complementary base pairing.

17. The method of claim 1, where the two orientations of the insert sequence are sequenced simultaneously.

18. The method of claim 1, where the two orientations of the insert sequence are sequenced in separate sequencing runs.

19-25. (canceled)

26. A method of making a sequencing library of nucleic acids comprising:
attaching first sequence tag to at least one end of an input fragment comprising an insert sequence to produce a tagged fragment, wherein the first sequence tag comprises sequence A;
amplifying the tagged fragment to produce a plurality of tagged fragments comprising the insert sequence, and at least some of the tagged fragments comprise a strand comprising a 5′ sequence tag comprising sequence A, wherein sequence A comprises a primer binding site;
amplifying the top strand of the tagged fragments with a primer set comprising primers of formulas C-A, and D-A to produce adaptor-tagged fragments, wherein sequences C and D are adaptor sequences;
wherein a first set of the adaptor-tagged fragments comprise a strand comprising 5′-end comprising sequences C and A, and the insert sequence; and
wherein a second set of the adaptor-tagged fragments comprises a strand comprising a 5′ end comprising sequences D and A, and the insert sequence.

27-49. (canceled)

50. A method of sequencing a library comprises adaptor-tagged fragments, the method comprising:
introducing first and second sets of the adaptor-tagged fragments to a solid support of a sequencing system,
wherein the first set comprises adaptor-tagged fragments of formula C-A-G-B-D and/or a complement thereof, and the second comprises adaptor-tagged fragments of formula D-A-G-B-C and/or a complement thereof, wherein sequences A and B comprise primer binding sites and molecular barcodes, sequences C and D are adaptor sequences, and G comprises a sequence of an input fragment, and
wherein the solid support comprising binding sites for one or more of sequences C, C′, D, and D′;
introducing a first set of sequencing primers to the solid support, wherein the first set comprises (a) sequencing primers that bind to sequence A and sequencing primers that bind to sequence B′, or (b) sequencing primers that bind to sequence A′ and sequencing primers that bind to sequence B;
sequencing the fragment sequences of the first and second sets of the adaptor-tagged fragments to obtain sequence reads from different orientations of the insert sequence simultaneously;
introducing a second set of sequencing primers which bind to regions downstream of (3′ to) the MBC;
determining complementary sequences of the molecular barcodes from different orientations of the adaptor-tagged fragments simultaneously;
analyzing the sequencing data to pair sequencing reads from different orientations of one of the insert sequences.

51-59. (canceled)
